(19) United States
(12) Patent Application Publication
(10) Pub. No.: US 2026/0221217 A1
(43) Pub. Date: Jul. 30, 2026

(54) DOMAIN ANNOTATION OF BIOLOGICAL MOLECULES USING MACHINE LEARNING MODELS

(71) Applicant: GDM Holding LLC, Mountain View, CA (US)

(72) Inventors: Felipe Llinares López, Paris (FR); Lucy Jane Colwell, Cambridge, MA (US); Laetitia Michele Catherina Papaxanthos, Horgen (CH)

(21) Appl. No.: 19/040,510

(22) Filed: Jan. 29, 2025

(51) Int. Cl. G16B 15/20; G16B 40/20

(52) U.S. Cl. CPC G16B 15/20; G16B 40/20

(57) ABSTRACT

Methods, systems, and apparatus, including computer programs encoded on a computer storage medium, for predicting domain annotations for a biological molecule. The system processes a network input that includes a representation of the biological sequence using an annotation neural network to generate a network output that defines a plurality of mask-annotation pairs. Each mask-annotation pair includes: (i) a mask that identifies a domain of the biological sequence; and (ii) an annotation that characterizes the domain of the biological sequence.

### BACKGROUND

This specification relates to processing data using machine learning models to predict domain annotations of biological molecules.

Machine learning models receive an input and generate an output, e.g., a predicted output, based on the received input. Some machine learning models are parametric models and generate the output based on the received input and on values of the parameters of the model. Neural networks are machine learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current value inputs of a respective set of parameters.

Biological molecules often include domains, which are distinct structural or functional regions of the molecule sequences. These domains play specific roles in the molecule's overall activity. For example, domains in protein sequences can be enzymatic active sites, DNA-binding regions, or transmembrane domains. In another example, domains in DNA and RNA sequences can include regulatory or coding regions that influence the molecules' function and interaction with other biological molecules.

### SUMMARY

This specification generally describes a system implemented as computer programs on one or more computers in one or more locations that can predict domain annotations of biological molecules using machine learning models.

In this specification, a biological molecule refers to a macromolecule that can be found in or introduced to living organisms and can include, for example, a protein molecule, a nucleic acid sequence of a deoxyribonucleic acid (DNA) molecule, or a nucleic acid sequence of a ribonucleic acid (RNA) molecule. These biological molecules are defined by sequences of building blocks or units, such as amino acids in proteins or nucleotides in nucleic acids. In this specification, the terms “biological molecule” and “biological sequence” are used interchangeably as the methods described herein analyze the sequence-level information of these molecules to infer their domain annotations.

In this specification, a “domain annotation” refers to the identification of the presence and classification of specific regions within a biological sequence. This region, referred to as a domain, represents a functional or structural unit within the biological sequence. A domain can be continuous, meaning it spans a single, uninterrupted segment of the sequence. For example, amino acids spanning positions 10-30 in a protein can form a continuous domain. A domain can also be discontinuous, meaning it includes multiple, non-overlapping segments of the sequence. For instance, amino acids spanning positions 10-20 and 40-50 may collectively form a discontinuous domain. Additionally, a domain can be repeated, meaning the same type of domain appears multiple times within the sequence. For example, a protein may have the same domain type present at positions 10-20 and again at positions 50-60. The described techniques can both identify a domain and infer the annotation of the domain. In implementations inferring the annotation of the domain can involve identifying a class of a set of classes to which the domain is predicted to belong, such as a set of structural classes that characterize a structure of the domain or a set of functional classes that characterize a function of the domain. Also or instead inferring the annotation of the domain can involve generating text, e.g. a caption, that describes the domain, which can be a more flexible way of characterizing a domain than assigning it to a class.

An “embedding” as used in this specification is an ordered collection of numerical values, e.g., a vector, array, or other tensor of numeric values, e.g., floating point values or other values, having a predetermined dimensionality. The space of possible embeddings having the predetermined dimensionality is referred to as the “embedding space.”

In one aspect, this specification describes a method for predicting domain annotations of a biological molecule. The method is implemented by a system including one or more computers. The system obtains data identifying a biological sequence, and processes a network input that includes a representation of the biological sequence using an annotation neural network to generate a network output that defines a plurality of mask-annotation pairs, where each mask-annotation pair includes: (i) a mask that identifies a domain of the biological sequence; and (ii) an annotation that characterizes the domain of the biological sequence. The system then outputs the plurality of mask-annotation pairs.

In some implementations, to process the network input to generate the network output, the system processes the representation of the biological sequence using an encoder block of the annotation neural network to generate a sequence of per-position embeddings that includes a respective per-position embedding for each position in the biological sequence, processes the sequence of per-position embeddings for the biological sequence using a decoder block of the annotation neural network to generate a predefined number of domain embeddings, and processes the domain embeddings using an output block of the annotation neural network to generate the plurality of mask-annotation pairs.

In some implementations, the representation of the biological sequence includes a respective initial per-position embedding of each position in the biological sequence that identifies a biological unit occupying the position in the biological sequence. To process the representation of the biological sequence using the encoder block of the annotation neural network to generate the sequence of per-position embeddings, the system processes the initial per-position embeddings of the positions in the biological sequence using self-attention operations update the initial per-position embeddings of the positions in the biological sequence.

In some implementations, to process the sequence of per-position embeddings for the biological sequence using the decoder block of the annotation neural network, the system initializes the predefined number of domain embeddings; and updates the domain embeddings by performing operations including: self-attention operations over the domain embeddings to update the domain embeddings; and cross-attention operations over the per-position embeddings for the biological sequence to update the domain embeddings.

In some implementations, to process the domain embeddings using the output block of the annotation neural network, for each domain embedding, the system processes the domain embedding to generate a score distribution over a set of classes, where the set of classes includes: (i) a plurality of domain classes, and (ii) a null class; selects a class from the set of classes based on the score distribution over the set of classes; and determines whether to generate a mask-annotation pair from the domain embedding based on the selected class from the set of classes.

In some cases, selecting a class from the set of classes based on the score distribution over the set of classes includes: selecting a class having a highest score under the score distribution over the set of classes. As another example a class can be sampled from the score distribution.

In some cases, for each domain embedding, determining whether to generate a mask-annotation pair from the domain embedding includes: determining that a mask-annotation pair should be generated from the domain embedding only if the selected class for the domain embedding is a domain class rather than the null class.

In some cases, for one or more domain embeddings, to select a class from the set of classes based on the score distribution over the set of classes, the system selects a domain class for the domain embedding. To process the domain embedding using the output block of the annotation neural network, the system processes the domain embedding and the sequence of per-position embeddings for the biological sequence to generate a score distribution over the biological sequence; generates a mask that identifies a domain of the biological sequence based on the score distribution over the biological sequence. The system outputs a mask-annotation pair that includes: (i) the mask that identifies the domain, and (ii) an annotation that specifies the domain class.

In some cases, for one or more of the domain embeddings, processing the domain embedding and the sequence of per-position embeddings for the biological sequence to generate the score distribution over the biological sequence includes, for each position in the biological sequence: determining a score for the position in the score distribution based on a measure of similarity between: (i) the domain embedding, and (ii) the per-position embedding for the position.

In some cases, for each position in the biological sequence, determining the score for the position in the score distribution based on the measure of similarity between: (i) the domain embedding, and (ii) the per-position embedding for the position includes: computing an inner product between the domain embedding and the per-position embedding for the position.

In some cases, processing the domain embeddings using the output block of the annotation neural network includes, for each domain embedding: processing the domain embedding to generate a sequence of text; and determining whether to generate a mask-annotation pair from the domain embedding based on the sequence of text.

In some cases, determining whether to generate the mask-annotation pair from the domain embedding based on the sequence of text includes: determining that a mask-annotation pair should be generated from the domain embedding only if the sequence of text indicates that the domain embedding corresponds to a domain in the biological sequence.

In some cases, for one or more domain embeddings, processing the domain embedding using the output block of the annotation neural network further includes: processing the domain embedding and the sequence of per-position embeddings for the biological sequence to generate a score distribution over the biological sequence; and generating a mask that identifies a domain of the biological sequence based on the score distribution over the biological sequence; and outputting a mask-annotation pair that includes: (i) the mask that identifies the domain, and (ii) an annotation that specifies the sequence of text generated from the domain embedding.

In some implementations, the plurality of mask-annotation pairs includes one or more mask-annotation pairs that each include a respective mask that identifies a discontinuous domain that includes multiple non-overlapping subsequences of the biological sequence.

In some implementations, the plurality of mask-annotation pairs includes multiple mask-annotation pairs that each include different masks but that share a same annotation.

In some implementations, the biological sequence has a length of at least 16.

In some implementations, the biological sequence includes an amino acid sequence of a protein molecule.

In some implementations, the biological sequence includes a nucleic acid sequence of a deoxyribonucleic acid (DNA) molecule.

In some implementations, the biological sequence includes a nucleic acid sequence of a ribonucleic acid (RNA) molecule.

In implementations of the described techniques the biological sequence is obtained from a biological sample, e.g. a sample of genetic material from a human, animal, plant, or other eukaryote, bacteria, or virus, e.g. using next-generation sequencing or other techniques.

In some implementations, the system further determines a function of the biological sequence based on the plurality of mask-annotation pairs. However this is not essential as it is biologically useful merely to identify a domain and characterize it, e.g., as a binding region of a protein (and hence a drug target), or as a regulatory region that controls expression of a gene (e.g. for use in identifying a molecular signature of a disease).

In some implementations, outputting the plurality of mask-annotation pairs includes storing the plurality of mask-annotation pairs in a memory, transmitting the plurality of mask-annotation pairs over a data communications network, or providing a representation of the plurality of mask-annotation pairs on a display of a user device.

In some implementations, the annotation neural network has been trained on a set of training examples by a machine learning training technique; wherein the set of training examples each include: (i) a training biological sequence, and (ii) a plurality of target mask-annotation pairs for the for the training biological sequence; and wherein training the annotation neural network on the set of training examples includes, for each training example, training the annotation neural network to reduce a discrepancy between: (i) a plurality of predicted mask-annotation pairs generated by processing the training biological sequence using the annotation neural network, and (ii) the plurality of target mask-annotation pairs specified by the training example.

In another aspect, this specification describes a system including one or more computers and one or more storage devices storing instructions that when executed by the one or more computers cause the one more computers to perform the operations of the method described above.

In another aspect, this specification describes one or more computer storage media storing instructions that when executed by one or more computers cause the one more computers to perform the operations of method described above.

The subject matter described in this specification can be implemented in particular embodiments so as to realize one or more of the following advantages.

Annotating biological molecule domains is essential for understanding the molecule's structure, function, and role in biological processes. Accurate domain annotations facilitate the discovery of new biological pathways, the identification of therapeutic targets, and the advancement of biotechnological applications. Accurate domain annotations are important for advancements in various fields, including drug discovery, disease understanding, and biotechnology.

Machine learning offers a scalable and efficient solution for predicting domains by analyzing large-scale biological data. Unlike traditional experimental methods, which are labor-intensive, machine learning models can process vast amounts of sequence data quickly and uncover complex patterns that may not be apparent through experimental analysis. However, using machine learning to predict domains poses several challenges. These include identifying discontinuous or repeated domains, ensuring the accurate delineation of domain boundaries, and minimizing false positives and negatives.

To address these issues, the system described in this specification leverages a machine learning model configured to generate mask-annotation pairs for biological sequences. These mask-annotation pairs provide detailed domain predictions by associating specific sequence regions (identified by the masks) with annotations that characterize the corresponding regions. This approach allows the system to predict overlapping domains, differentiate between repeated and discontinuous domains, and provide accurate boundaries without the need for post-processing.

In some implementations, the system uses a prediction machine learning model that includes an encoder network and a decoder network. The encoder generates context-aware embeddings for each position in the molecule sequence, and the decoder predicts domain embeddings that are processed into mask-annotation pairs. This architecture enables the system to efficiently capture long-range dependencies in biological sequences, improving prediction accuracy and scalability. The system also benefits from transformer-based self-attention and cross-attention mechanisms, which enhance its ability to handle complex domain structures.

Leveraging the paired mask-annotation prediction and the encoder-decoder network architecture, as described above, enables the system to predict domain annotations more accurately than conventional systems, which often struggle with overlapping, discontinuous, or repeated domains.

Furthermore, the described techniques predict the mask-annotation pairs based on a predefined number of embeddings. The number of embeddings is independent of the length of the biological sequence, and thus can be significantly shorter than the length of the biological sequence. For example, the system can generate 50 domain embeddings for a biological sequence of length 1000. The architectural design of the prediction model leverages the domain embeddings to achieve several improvements, as described below.

An improvement in computer technology is achieved by reducing the consumption of computational resources. Attention operations enable the generation of rich embeddings through fine-grained and long-range information sharing. However, self-attention operations over the per-position embeddings are computationally expensive because their complexity scales quadratically with the number of per-position embeddings. The techniques described herein addresses this issue by performing self-attention over the domain embeddings and cross-attention over the per-position embeddings to update the domain embeddings. This approach partially decouples the computational complexity of attention operations from the length of the biological sequence, thereby significantly reducing computational resource consumption.

An improvement in machine learning technology is also achieved because domain embeddings provide a natural solution to the problem of predicting overlapping or discontinuous domains. Specifically, each domain embedding is projected to generate a respective mask-annotation pair. If a domain embedding is mapped to a null class, it is simply discarded. This mechanism ensures efficient and accurate handling of complex domain structures, including overlapping or discontinuous regions, enhancing the flexibility and applicability of the model.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example machine learning system.

FIG. 2A shows the operations of an example of the annotation system.

FIG. 2B shows the operations of an example of the output block.

FIG. 2C shows the operations of another example of the output block.

FIG. 3 is a flow diagram of an example process for predicting domain annotations of a biological molecule sequence.

FIG. 4 is a flow diagram of an example process for training an annotation neural network.

FIG. 5 demonstrates the performance of the annotation system.

Like reference numbers and designations in the various drawings indicate like elements.

### DETAILED DESCRIPTION

Determining the domains of biological molecules is crucial for understanding their structure, function, and roles in biological processes. This specification describes techniques for predicting domain annotations of biological molecules using machine learning models. By accurately identifying domains, these techniques provide insights into the functional characteristics and interactions of biological molecules.

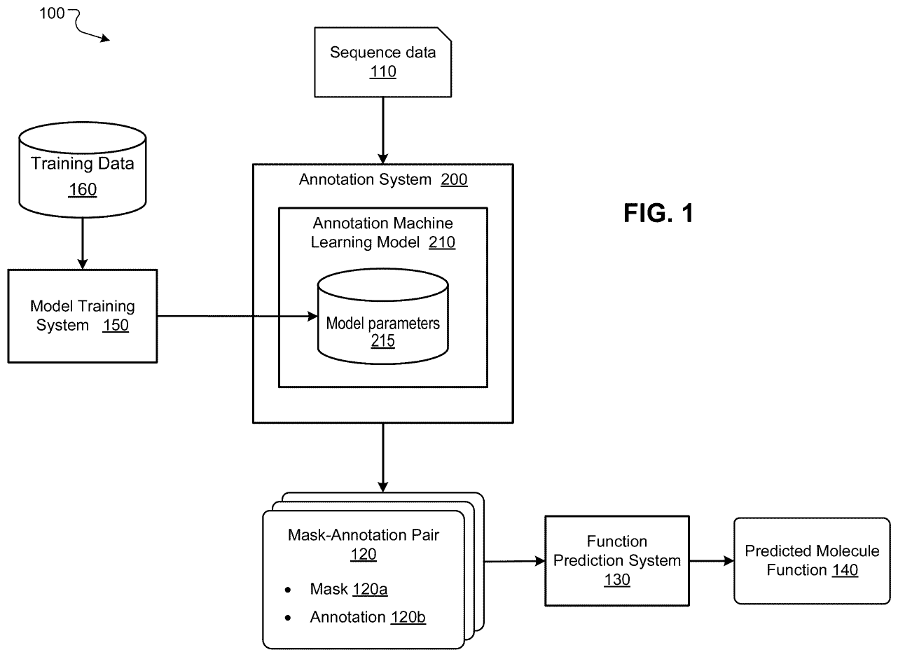

FIG. 1 shows an example machine learning system 100. The machine learning system 100 is an example of a system implemented as computer programs on one or more computers in one or more locations in which the systems, components, and techniques described below are implemented.

The system 100 includes an annotation system 200 that processes sequence data 110 identifying a biological sequence to generate output data 120 that defines a set of mask-annotation pairs 120. Each mask-annotation pair includes (i) a mask 120a that identifies a domain of the biological sequence, and (ii) an annotation 120b that characterizes the domain of the biological sequence.

As will be described below, the mask 120a and the annotation 120b can be in any appropriate format. For example, the mask 120a can be a binary vector indicating the positions of the domain in the sequence or a set of points representing the boundaries of the domain. The annotation 120b can be a categorical label, a textual description, or a combination thereof.

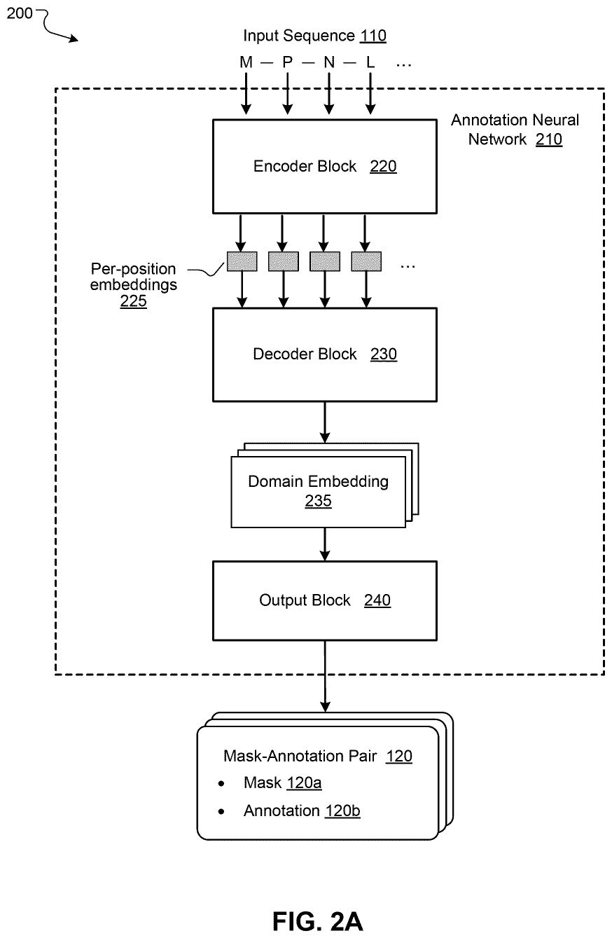

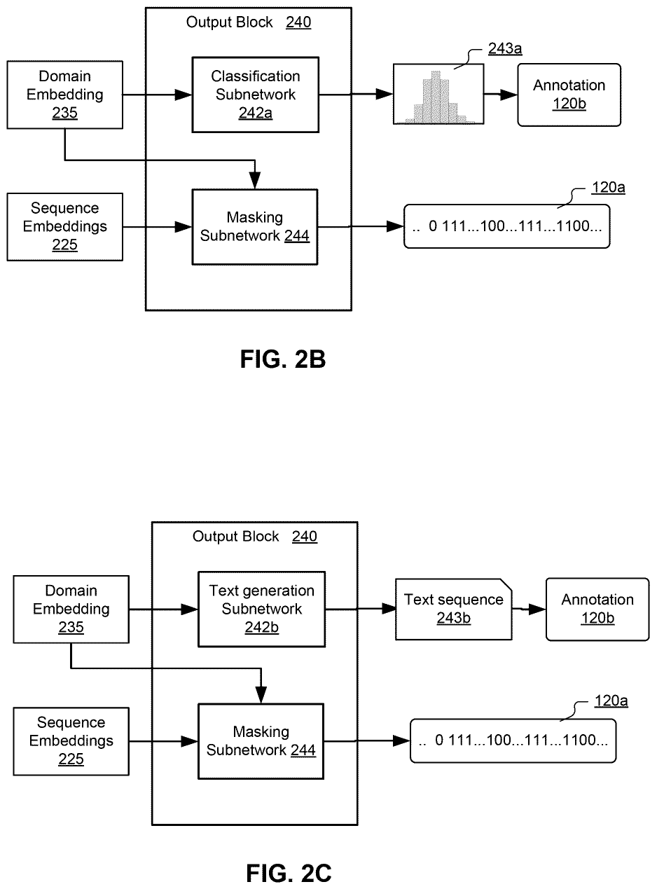

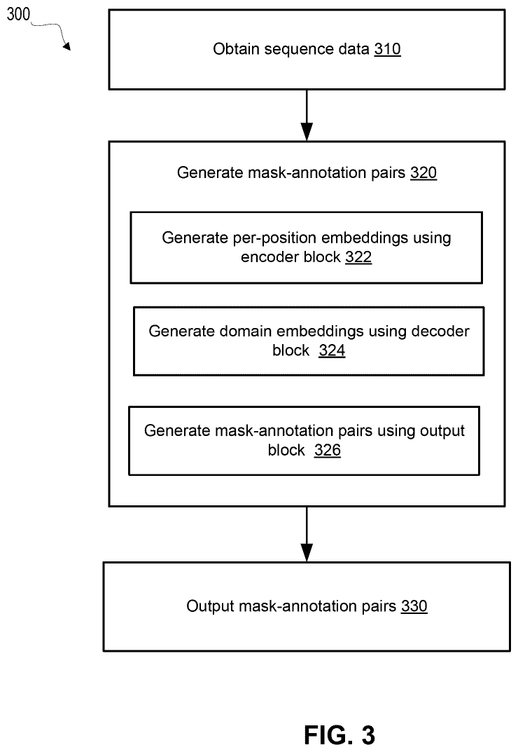

In particular, the annotation machine learning model 230 can be a neural network that includes multiple layers designed to capture and process the relationships in the sequence data 110. Operations of the annotation system 200 and example architecture of the annotation neural network will be described with references to FIGS. 2 and 3. In general, the annotation system 200 aims to predict an appropriate number of mask-annotation pairs to effectively represent the domains in a given biological sequence. The number of mask-annotation pairs can be selected based on the specific application or dataset. For example, if the sequence length and expected number of domains are known, the system can determine the total number of mask-annotation pairs by considering the maximum number of domains in similar sequences.

The output of the mask-annotation pairs allows the system to annotate various types of domains, including continuous domains, discontinuous domains, and repeated domains. This flexibility enables comprehensive annotation of complex sequences, including those with overlapping or discontented domains. By accurately distinguishing between different types of domains, the system improves the accuracy of domain annotation, leading to improved functional insights and enhanced downstream applications such as protein function prediction and structural modeling.

In some cases, the annotation system 200 can be used to generate domain annotations of protein sequences. In these cases, the sequence data 110 specifies a protein sequence. A protein sequence can be represented as a sequence of amino acid residues, each encoded by a single-letter or three-letter code according to standard biochemical conventions (FIG. 2A illustrates one-letter amino acid codes). The output mask-annotation pairs specify regions of the protein sequence that correspond to specific domains, along with annotations that characterize these domains. Examples of protein domains include (1) structural domains, which define the three-dimensional structure of a protein, such as secondary structural domains, e.g. alpha helices, beta sheets, and loops, and (2) functional domains, which define the function of a protein, such as catalytic domains, binding domains, and regulatory domains.

In some cases, the annotation system 200 can be used to generate domain annotations of DNA or RNA sequences. A DNA or RNA sequence can be represented by a sequence of nucleotides, each encoded by a single-letter code corresponding to the four primary bases: adenine (A), cytosine (C), guanine (G), and thymine (T) for DNA, or uracil (U) for RNA in place of thymine.

In some implementations, DNA or RNA coding sequences can be represented by codon sequences instead of nucleotide sequences. A codon is a triplet of nucleotides that encodes for a specific amino acid or a stop signal during translation. Representing a coding sequence by its series of codons can provide additional information about the potential amino acid sequence it encodes.

The output mask-annotation pairs 120 generated by the annotation system for a DNA or RNA sequence can identify regions within the sequence that correspond to functional elements, such as promoter regions, enhancers, splice sites, or conserved secondary structures like stem-loops. These annotations can also specify regions involved in coding for functional proteins or regulatory RNAs, offering valuable insights into the roles of specific nucleotide sequences within the broader genomic or transcriptomic context.

The system 100 can further include a function prediction system 130 configured to predict the functions and/or properties of the biological molecule from the mask-annotation pairs 120 predicted by the annotation system. In some cases, the function prediction system 130 can use a predefined function that maps combinations of domain annotations to corresponding functions or properties. For example in a protein, a combination of a catalytic domain and a regulatory domain could be assigned a function combining enzymatic activity and regulation; kinase and SH2 could relate to signal transduction; a zinc finger and homeodomain could relate to DNA binding and transduction, and so forth. In DNA the combinations could relate to combinations of one or more of motifs, repeat elements, epigenetic marks, and structural features. As some examples, a combination of a TATA box and a CAAT box could indicate a gene with expression regulated in response to particular signals, a combination of a short tandem repeat and an Alu element could indicate presence of a disease, or a combination of a repeat-spacer array and a Cas protein motif could indicate presence of a CRISPR/Cas system, and so forth. Many such combinations are known and can, e.g., be included in a function dictionary. Also or instead, the function prediction system 130 can use a machine learning model to predict the functions and/or properties of the biological molecule. The machine learning model can be trained on labeled datasets of biological molecules, where the inputs are domain annotations and the outputs are known functions or properties.

The predicted functions or properties can be used in various applications. For instance, for proteins, the predicted functions can aid in understanding the role of a protein in signaling pathways, identifying potential therapeutic targets, or predicting the binding affinity of a molecule to other biomolecules. Similarly, for DNA or RNA sequences, predicted functions can inform gene regulation mechanisms, identify functional non-coding regions, or guide the design of synthetic sequences for gene editing or therapeutic purposes.

Below are a few additional examples of the downstream tasks that can be performed based on the domain annotations generated by the annotation system.

The domain annotations generated by the annotation system can be used in drug target discovery. For example, given novel proteins that are highly expressed in tumor cells, e.g. from experimental assays (such as immunoassays, proteomic profiling, functional screening, or other techniques), the annotation system can perform protein domain annotation for one or more candidate proteins. Based on the annotation results, one or more of the candidate proteins can be identified as drug targets, e.g., because the protein includes a kinase domain typically associated with phosphorylation signaling pathways known to drive tumor growth. By identifying such proteins, e.g. the kinase domains of such proteins, as drug targets, the system supports the discovery and development of targeted therapies.

The domain annotations generated by the annotation system can also be used in disease identification or disease variant interpretation. For example, some variants in DNA, RNA or protein sequences in a particular annotated domain can be linked to disease and/or can be used to identify a particular drug that should be used to treat a disease (personalized medicine). In clinical genomics, functional annotation helps interpret whether genetic variants in patients affect crucial domains and cause disease. For example, loss-of-function mutations in DNA-repair domains may predispose patients to cancer. The annotation system can aid in identifying such critical mutations, thereby providing insights into disease mechanisms and supporting precision medicine initiatives. More generally, variants in an identified and annotated domain are more likely than others to disrupt functions and cause disease. For example, as previously described, a variant in a kinase domain could drive oncogenesis; a variant in an SH3 domain could alter signaling pathways in cancer or other disorders; or a variant in an immunoglobulin domain could result in an immune deficiency. As some further examples a variant in a DNA promotor or enhancer region, or generally a mutation or structural change in a domain associated in gene expression, e.g. transcription or regulation, could affect cancer tumor growth or suppression; or a mutation or structural variation in RNA, e.g. a change in a secondary structure such as an RNA-stem-loop, could affect transcription. As previously described, it is not essential for the biological function of an annotated domain, or combination of domains to be known, but where it is known, this can then aid in identification or treatment of a particular disease.

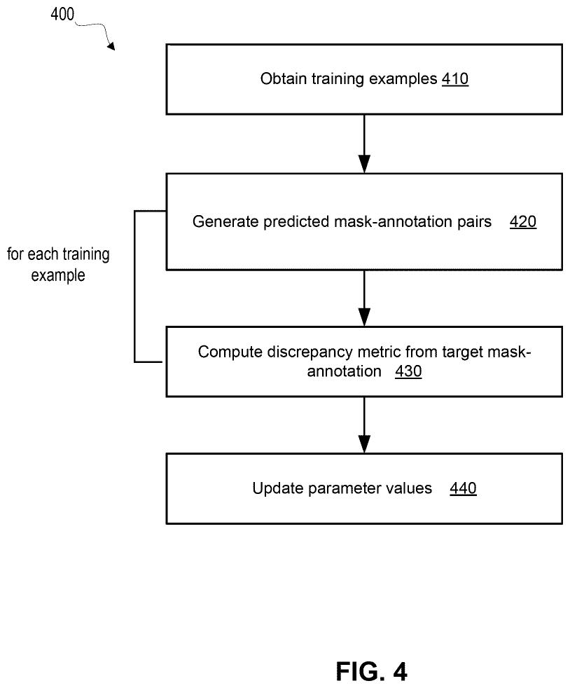

The system 100 further includes a model training system 150 that determines the parameters 235 of the annotation machine learning model 230 based on training data 160. Operations of the training system 150 will be described with reference to FIG. 4.

FIG. 2A shows the operations and data flow of an example of the annotation system 200. The system 200 is an example of a system implemented as computer programs on one or more computers in one or more locations, in which the systems, components, and techniques described below can be implemented.

The system receives input sequence data 110 that defines the sequence of a biological molecule. As described above, sequence data 110 can define an amino acid sequence of a protein molecule, or a nucleotide sequence or a codon sequence of a DNA or RNA molecule.

Before processing the input sequence data 110 using the annotation neural network 210, the system 200 can convert the input sequence into a numerical representation. For example, the system can use one-hot encoding, which represents each element in the sequence (e.g., an amino acid, nucleotide, or codon) as a binary vector where a single “1” indicates the presence of the element, and all other positions are “0”. Alternatively, as described below, the system can use embedding techniques to map each element in the sequence to a dense, low-dimensional vector representation.

The annotation neural network 210 can adopt any appropriate architecture suitable for processing sequence data to predict the domain annotation. In the example shown in FIG. 2A, the annotation neural network 210 includes an encoder block 220, a decoder block 230, and an output block. Each network block is a sub-network within the annotation neural network 210 and includes a plurality of layers. The encoder block 220 is configured to process a representation of the biological sequence to generate a sequence of per-position embeddings 225. The decoder block 230 is configured to receive the per-position embeddings 225 generated by the encoder block 220, and process the per-position embeddings 225 to generate a set of domain embeddings 235. The output block 240 is configured to process an input including the domain embeddings 235 to generate the set of mask-annotation pairs 120.

The encoder block 220 can adopt any suitable architecture. In some implementations, the encoder block 220 includes one or more attention layers configured to implementing attention mechanisms. In some implementations, the encoder block 220 includes one or more convolutional layers configured to implement convolutional operations. The encoder block 220 is configured to process a sequence of initial per-position embeddings derived from the input sequence 110 and update them into contextualized per-position embeddings 225. In general an attention mechanism can compute query, key and value embeddings from elements of one or more attention layer inputs, and can compute a weighted sum of the value embeddings with weights depending on a similarity between the query and key embeddings, to determine the attention layer output.

The number of per-position embeddings 225 corresponds to the length of the biological sequence 110. Each per-position embedding is a numerical representation, such as a vector, e.g. of floating-point values, in a pre-defined vector space, that represents the element occupying the corresponding position in the input sequence 110. In principle the embedding of each nucleotide in a DNA or RNA sequence, or of each codon or each amino acid in an amino acid sequence, can be a one-hot embedding. In some implementations the embeddings are pretrained or learned embeddings in a vector space, e.g. from an embedding neural network. The dimension of each per-position embedding can depend on factors such as the complexity of the biological sequence, the desired level of detail in the representation, and computational resource constraints.

The initial per-position embeddings can be generated by mapping each element in the input sequence 110 to a dense vector representation using an embedding neural network layer or a lookup table. In some cases, the initial per-position embeddings are augmented with positional information to ensure that the encoder block can account for the order of elements in the sequence. For example, the positional information can be incorporated by appending a positional encoding vector to the respective initial per-position embedding. The positional encoding vector can be generated in any suitable way, e.g. using a frequency-based positional embedding such as a function, e.g., a sinusoidal function of the position of the element in the sequence.

The attention layers of the encoder block 220 are configured to capture relationships between different positions in the input sequence 110. Specifically, the attention layers implement a self-attention mechanism, which causes the initial per-position embedding at each position to attend to the initial per-position embeddings at all positions of the input sequence to generate the updated per-position embeddings. This process allows the encoder block to capture both local and long-range dependencies in the sequence, and generate contextualized embeddings that reflect the relationships and interactions across the biological sequence.

The decoder block 230 is configured to receive the per-position embeddings 225 generated by the encoder block 220 and process them to generate a set of domain embeddings 235. The set of domain embeddings 235 correspond to a set of potential domains that the input sequence 110 may contain. These domain embeddings 235 represent high-level latent features associated with specific regions or patterns in the sequence that align with potential functional or structural domains. The number of domain embeddings 235 corresponds to the number (N) of the output mask-annotation pairs 120, which can be pre-selected, e.g., based on the maximum number of domains the model is designed to predict for a given type of sequences.

In some implementations, the decoder block 230 includes one or more attention layers configured to update an initial set of domain embeddings using attention mechanisms. In some cases, the initial set of domain embeddings can be initialized as a set of learnable vectors, where the component values of these embeddings are updated as trainable parameters during training. At inference, the initial set of domain embeddings are initialized to their trained values.

In some cases, the attention layers of the decoder block 230 include both (i) self-attention layers configured to perform self-attention operations and (ii) cross-attention layers configured to perform cross-attention operations. The self-attention operations cause each domain embedding to attend to all other domain embeddings, allowing the decoder to capture relationships and dependencies across different domain embeddings. The cross-attention operations cause each domain embedding to attend to the per-position embeddings 225 for all positions, allowing the decoder to integrate position-specific information from the sequence into each domain embedding. By combining self-attention and cross-attention operations, the decoder block 230 refines the initial domain embeddings into updated domain embeddings 235 that are contextually enriched and position-aware.

As an example, the decoder block 230 can generate one domain embedding, e.g. a d-dimensional vector, for each mask-annotation pair of the set of mark-annotation pairs to be generated. The decoder block 230 can arrange the self-attention layers, the cross-attention layers, as well as other types of network layers, such as feedforward layers and normalization layers, in any appropriate manner. Example implementations of the decoder block are described in Carion et al., “End-to-End Object Detection with Transformers,” European conference on computer vision, pages 213-229, Springer, 2020. As one illustrative example, the decoder block 230 can comprise a transformer neural network, i.e. one that includes a neural network with one or more transformer blocks, each transformer block including an attention layer that can be followed by a feedforward neural network layer. The transformer neural network can process N query inputs (e.g. each a learned embedding, fixed during operation of the system), and key-value embedding pairs derived from the per position embeddings 235, to generate N d-dimensional vector outputs for the output block 240.

The output block 240 is configured to process an input including the domain embeddings 235 to generate the set of mask-annotation pairs 120. The output block 240 can have any appropriate network architecture. Examples of operations of the output block 240 are described below with references to FIGS. 2B and 2C.

As described below, the mask-annotation pairs 120 enable the system 200 to represent repeated and/or discontinued domains in certain implementations. In some cases, the system further performs a filtering process on the generated mask-annotation pairs 120 to remove certain annotations representing overlapping domains. For example, the system 200 can cluster overlapping masks that share the same annotation, and retain only the mask with the highest confidence score, e.g., computed using a score distribution determined for the corresponding domain embedding over the input sequence. Examples of determining the score distribution over the input sequence are described below with reference to FIG. 2B.

The system 200 can output mask-annotation pairs 120 in a variety of ways, such as storing the plurality of mask-annotation pairs 120 in a memory, transmitting the plurality of mask-annotation pairs over a data communications network, or providing a representation of the plurality of mask-annotation pairs on a display of a user device. As described with reference to FIG. 1, the mask-annotation pairs 120 can be used in a downstream task such as predicting functions or properties of the input sequence 110.

FIG. 2B shows the operations of an example of the output block 240. The output block 240 includes (i) a classification subnetwork 242a configured to determine a class for each domain embedding 235 generated by the decoder block 230, and (ii) a masking subnetwork 244 configured to generate a mask 120a for each domain embedding 235 that has been determined to be associated with a valid domain class. The annotation 120b is determined from the domain class determined by the classification subnetwork 242a. The following operations are described for a particular domain embedding 235. These operations are performed for all the domain embeddings generated by the decoder block 230. The classification subnetwork 242a and masking subnetwork 244 can be particular to each domain embedding or shared between the domain embeddings 235.

The classification subnetwork 242a processes the domain embedding 235 to generate a score distribution 243a over a set of classes. The set of classes includes: (i) a plurality of domain classes, and (ii) a null class. Each domain class represents a type of domain that can be found in the input biological sequence. The plurality of domain classes can be pre-defined based on known domain types for the type of biological sequence. The null class represents the absence of a domain, that is, when a particular domain embedding 235 is not associated with any valid domain type. The introduction of a null class allows the model to predict that a particular domain embedding does not correspond to any valid domain. This benefits the model by enabling it to dynamically adjust the number of predicted domains for each sequence, effectively reducing false positive predictions and improving the precision of the domain annotations.

The score distribution 243a represents the probabilities of the domain embedding 235 belonging to each class in the set of classes, as determined by the classification subnetwork. The system 200 can select a class for domain embedding 235 from the set of classes based on the score distribution 243 over the set of classes. For example, the system can select a class that has the highest score under the score distribution over the set of classes as the class associated with the domain embedding 235. The system can use the selected class to generate the annotation 120b for the domain embedding 235, e.g., as a text label or a numerical value corresponding to the selected class. The annotation 120b specifies the type of domain.

The classification subnetwork 242a can have any appropriate network architecture. In a particular example, the classification subnetwork 242a includes a linear layer followed by a softmax layer.

Based on the selected class, the system can determine whether to generate the mask-annotation pair 120 for the domain embedding 235. In particular, the system can determine to generate the mask-annotation pair 120 only if the selected class for the domain embedding 235 is a valid domain class rather than the null class.

Once it is determined that the mask-annotation pair 120 is to be generated for the domain embedding 235, the system uses the masking subnetwork 244 to process (i) the domain embedding 235 and (ii) the sequence of per-position embeddings 225 generated by the encoder block 220 to generate a mask 120a for the domain embedding 235. For example the masking subnetwork 244 can process the sequence of per-position embeddings 225 conditioned on the domain embedding 235 to generate a corresponding output sequence; and can have any suitable neural network architecture, e.g. feed forward, convolutional, attention-based, and so forth.

As described above, in some cases, the mask 120a can be a binary vector indicating the positions in the input sequence 110 that belong to the domain. For example, the binary vector can have the same length as the input sequence, and each element of the vector can have a value of 1 if the corresponding position in the input sequence is part of the domain and a value of 0 otherwise.

In some implementations, the masking subnetwork 244 generates a score distribution over the input sequence 110 and determines the mask 120a based on the score distribution. The score distribution includes, for each position in the input sequence, a respective score indicating a probability of the position belonging to the domain. The mask 120a can be determined, e.g., by applying a threshold to the score distribution, where positions with scores above the threshold are assigned a value of 1, and positions with scores below the threshold are assigned a value of 0. Since the system determines whether each individual position belongs to a domain, a discontinuous domain can be effectively represented. In this case, the mask identifies multiple non-overlapping subsequences of the biological sequence.

Also or instead, the respective score at each position can be determined based on a measure of similarity between: (i) the domain embedding 235, and (ii) the per-position embedding 225 for the position. That is, the score reflects how well the characteristics of the domain embedding align with the features of the per-position embedding at that specific position. Any appropriate type of similarity measure can be used. In a particular example, the similarity measure is computed as an inner product between the domain embedding 235 and the per-position embedding 225 for the position. In some cases, before computing the similarity measure, the domain embedding 235 is further processed, e.g., via a linear transformation, to project it into a space that aligns with the representation space of the per-position embeddings 225.

The operations of class selection and annotation generation are performed for each domain embedding 235. In some cases, the same class can be selected for multiple different domain embeddings, which results in multiple mask-annotation pairs 120 that each include different masks but that share the same annotation. This facilitates the system representing repeated domains, which are domains that occur multiple times in the input sequence.

FIG. 2C illustrates the operations of another example of the output block 240. This example is similar to the one described with reference to FIG. 2B, except that the classification subnetwork is replaced with a text generation network 242b configured to process the domain embedding 235 to generate a text sequence 243b that characterizes the domain embedding 235. In some cases, the text sequence 243b provides a descriptive annotation of the domain and can be used directly as the annotation 240b. In some cases, the text sequence 243b can be further processed, e.g., by applying natural language processing techniques, filtering, or mapping to a predefined taxonomy, to generate the final annotation 240b for the domain.

The text generation network 242b can have any appropriate architecture for a text generative network. For example, the text generation network 242b can be implemented as a transformer-based architecture, or use recurrent neural network (RNN) architectures, such as Long Short-Term Memory (LSTM) or Gated Recurrent Unit (GRU) networks.

In some cases, the text generation network 242b can be an autoregressive large language model, e.g., by implementing the Transformer architecture described in Vaswani, A. “Attention is all you need.” Advances in Neural Information Processing Systems (2017). The large language model can be pretrained to perform a language modeling task, e.g., a next character prediction task, prior to being fine-tuned along with the rest of the annotation neural network. Such text generation network 242b can be conditioned on the domain embedding 235 either as side information provided to the language model, or by providing the domain embedding in the form of one or more soft tokens as part of the (autoregressive) input sequence to the model, e.g. as a prompt to steer the text generation. Using a text generation network offers several advantages. It can make the predictions more easily understandable without having to map them back to their description. It can also potentially discover new functional domains that do not exist in the training set. Such a system can be trained as previously described, but using training data that comprises text descriptions of domains.

Based on the generated text sequence 243b, the system can determine whether to generate the mask-annotation pair 120 for the domain embedding 235. For example, the system can determine to generate the mask-annotation pair 120 only if the text sequence 243b corresponds to a valid domain class. This determination can be made by comparing an embedding of the text sequence 243b with an embedding of text representing a null class or a predefined set of valid domain classes. For example, if the embedding of the text sequence matches that of a null class, the mask-annotation pair 120 will not be generated to represent the domain.

FIG. 3 is a flow diagram of an example process 300 for predicting domain annotations of a biological molecule sequence. For convenience, the process 300 will be described as being performed by a system of one or more computers located in one or more locations. For example, an annotation system, e.g., the annotation system 200 of FIG. 2A, appropriately programmed in accordance with this specification, can perform the process 300.

At 310, the system obtains data identifying a biological sequence. As described with references to FIG. 1 and FIG. 2A, the biological sequence can be a protein molecule, a DNA molecule, or an RNA molecule. The biological sequence can have any appropriate length. In an illustrative example, the biological sequence has a length of at least 16.

At 320, the system processes a network input using an annotation neural network to generate a network output that defines a plurality of mask-annotation pairs. The network includes a representation of the biological sequence, e.g., as a numerically encoded vector of the biological sequence. Each mask-annotation pair includes (i) a mask that identifies a domain of the biological sequence, and (ii) an annotation that characterizes the domain of the biological sequence. In some cases, the set of mask-annotation pairs can include one or more mask-annotation pairs that each include a respective mask that identifies a discontinuous domain that comprises multiple non-overlapping subsequences of the biological sequence. In some cases, the set of mask-annotation pairs includes multiple mask-annotation pairs that each include different masks but that share the same annotation. The number of the mask-annotation pairs can depend on the type of sequence as well as the particular sequence. In some cases, the set of mask-annotation pairs includes at least 10 mask-annotation pairs. In some other cases, the set of mask-annotation pairs includes fewer than 10 mask-annotation pairs.

As described with reference to FIG. 2A, in some implementations, the annotation neural network includes an encoder block, a decoder block, and an output block. At 322, the system processes the representation of the biological sequence using the encoder block to generate a sequence of per-position embeddings that includes a respective per-position embedding for each position in the biological sequence. At 324, the system processes the sequence of per-position embeddings for the biological sequence using the decoder block to generate a predefined number of domain embeddings. At 326, the system processes the domain embeddings using an output block of the annotation neural network to generate the plurality of mask-annotation pairs. Example processes of 326 will be described below.

As described with reference to FIG. 2B, in some implementations, the system processes the domain embedding to generate a score distribution over a set of classes. The set of classes includes (i) a plurality of domain classes, and (ii) a null class. The system selects a class from the set of classes based on the score distribution over the set of classes, e.g., by selecting a class having the highest score under the score distribution over the set of classes. The system determines whether to generate a mask-annotation pair from the domain embedding based on the selected class from the set of classes. For example, the system can determine that a mask-annotation pair should be generated from the domain embedding only if the selected class for the domain embedding is a domain class rather than the null class. To generate the mask for a domain with a valid domain class, the system processes the domain embedding and the sequence of per-position embeddings for the biological sequence to generate a score distribution over the biological sequence, and generates a mask that identifies a domain of the biological sequence based on the score distribution over the biological sequence.

As described with reference to FIG. 2C, in some implementations, the system processes the domain embedding to generate a sequence of tex. For example, the system can determine that a mask-annotation pair should be generated from the domain embedding only if the sequence of text indicates that the domain embedding corresponds to a domain in the biological sequence. To generate the mask for a domain with a valid domain class, the system processes the domain embedding and the sequence of per-position embeddings for the biological sequence to generate a score distribution over the biological sequence, and generates a mask that identifies a domain of the biological sequence based on the score distribution over the biological sequence.

At 330, the system outputs the mask-annotation pairs. As described above with reference to FIG. 2A, The system can output mask-annotation pairs in a variety of ways, such as storing the plurality of mask-annotation pairs in a memory, transmitting the plurality of mask-annotation pairs over a data communications network, or providing a representation of the plurality of mask-annotation pairs on a display of a user device. As described with reference to FIG. 1, the system or another system can use the mask-annotation pairs to perform a downstream task such as predicting functions or properties of the input sequence.

FIG. 4 is a flow diagram of an example process for training an annotation neural network. For convenience, the process 400 will be described as being performed by a system of one or more computers located in one or more locations. For example, a training system, e.g., the model training system 150 of FIG. 1, appropriately programmed in accordance with this specification, can perform the process 400.

At 410, the system obtains a set of training examples. Each training example includes (i) a training biological sequence, and (ii) a plurality of target mask-annotation pairs for the for the training biological sequence. The training examples can be obtained based on a variety of sources, such as publicly available biological sequence databases (of which there are many), or curated datasets containing manually annotated domains. Examples of publicly available protein domain databases include UniProtKB (Universal Protein Knowledgebase, www.uniprot.org) which includes functional and structural domain annotations; and InterPro (www.ebi.ac.uk/interpro). Examples of publicly available DNA domain databases include the UCSC Genome Browser (US National Institutes of Health; genome.ucsc.edu); and the ENCODE database (Encyclopedia of DNA Elements; www.encodeproject.org) which includes functional and structural domain annotations. Examples of publicly available RNA domain databases that include functional and structural domain annotations are Rfam (rfam.org); GtRNAdb (Transfer RNA Database; gtrnadb.ucsc.edu; and RNAcentral (rnacentral.org). In some cases, the training examples can be augmented with synthetic data generated by perturbing real sequences or creating simulated annotations to improve model generalization. The training examples can be preprocessed to align with the input and output formats expected by the neural network.

The system performs 420 and 430 for each training example.

At 420, the system processes the training biological sequence using the annotation neural network with the current values of model parameters to generate a predicted set of mask-annotation pairs.

At 430, the system computes the value of a discrepancy measure between: (i) the predicted set of mask-annotation pairs, and (ii) the plurality of target mask-annotation pairs specified by the training example.

The discrepancy measure can be computed in any appropriate manner that measures the difference between the predicted annotations and the target truth annotations. In some implementations, a Hungarian algorithm is used to identify the optimal assignment that assigns the predicted mask-annotation pairs to the mask-annotation pairs. The Hungarian algorithm computes a cost matrix for all possible pairings of predicted and target pairs, identifies the permutation of assignments that minimizes a total cost, and matches each target pair to at most one predicted pair and vice versa. The computes the discrepancy measure based on the optimal assignment.

In a particular example, for a target mask-annotation pair Yₖ=(cₖ, mₖ) and a predicted mask-annotation pair Ý_{k′}=(ĉ_{k′}, m_{k′}), the Hungarian algorithm finds an optimal assignment by finding the optimal permutation \hat{σ} that minimises the total assignment cost, that is,

$$σ = arg min {\sum }_{k = 1 , {c}_{k} \neq ∅}^{N} {L}_{match} ( {Y}_{k} , {\hat{Y}}_{σ ( k )} ) .$$

The matching cost is computed as L_{match}(Yₖ,Ŷ_{σ(k)})=−\hat{p}_{σ(k)}(cₖ)+Lₘₐₛₖ(\hat{m}_{σ(k)},mₖ), where Lₘₐₛₖ is a linear combination of a focal loss and a dice loss. Here, assignments to the null class Ø do not appear in the Hungarian cost. Where the number of domains in a target mask-annotation pair is less than N the target can be padded with null classes.

After the optimal matching is identified, the loss function using the optimal permutation \hat{σ} is computed as

$${L}_{mask - cls} ( Y , \hat{Y} ) = {\sum }_{k = 1 , {c}_{k} \neq ∅}^{N} ( - log ( {\hat{p}}_{\hat{σ} ( k )} ( {c}_{k} ) ) + {L}_{mask} ( {\hat{m}}_{\hat{σ} ( k )} , {m}_{k} ) ) + w {\sum }_{k = 1 , {c}_{k} \neq ∅}^{N} ( - log ( {\hat{p}}_{\hat{σ} ( k )} ( ∅ ) ) ) .$$

w is a weight parameter for the contribution of incorrect assignments to the null class. Varying the weight w can be used to adjust the precision-coverage trade-off. A small value for w, e.g., w=0.1, can be used to increase coverage, allowing the model to identify more domains at the expense of slightly reduced precision. An increased value for w, e.g., w=0.4, can be used to prioritize precision by reducing false positives but may decrease coverage.

At 430, the system updates the current values of a set of neural network parameters of the annotation neural network using the gradients of the loss function using the update rule of an appropriate gradient descent optimization technique, e.g., RMSprop or Adam.

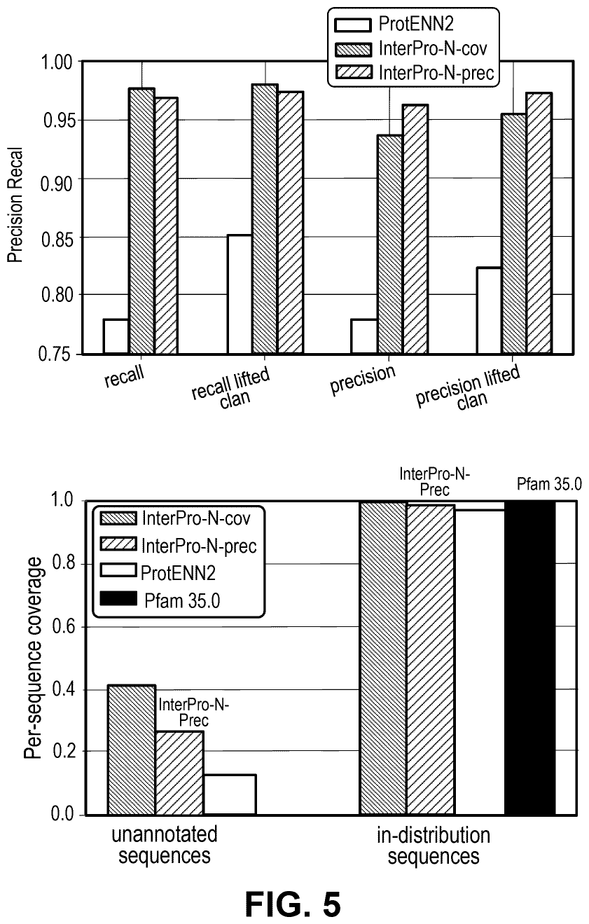

FIG. 5 demonstrates the performance of the annotation system for generating domain annotations for protein sequences. In particular, FIG. 5 compares the performances of (i) “InterPro-N-cov”, which uses the annotation neural network trained with the weight parameter w=0.1, (ii) “InterPro-N-prec”, which uses the annotation neural network trained with the weight parameter w=0.4, and (iii) “ProtENN2,” which is a previous state-of-the-art method.

As shown in FIG. 5, the techniques described herein achieve significant improvements of more than 15% in precision and recall compared to ProtENN2. Additionally, compared to ProtENN2, the annotation neural network provides higher per-sequence coverage in both the in-distribution set and the set of sequences without annotations.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more computers, the method comprising:
obtaining data identifying a biological sequence;
processing a network input that comprises a representation of the biological sequence using an annotation neural network to generate a network output that defines a plurality of mask-annotation pairs, wherein each mask-annotation pair comprises:
(i) a mask that identifies a domain of the biological sequence; and
(ii) an annotation that characterizes the domain of the biological sequence; and
outputting the plurality of mask-annotation pairs.

2. The method of claim 1, wherein processing the network input that comprises the representation of the biological sequence using the annotation neural network to generate the network output that defines the plurality of mask-annotation pairs comprises:
processing the representation of the biological sequence using an encoder block of the annotation neural network to generate a sequence of per-position embeddings that includes a respective per-position embedding for each position in the biological sequence;
processing the sequence of per-position embeddings for the biological sequence using a decoder block of the annotation neural network to generate a predefined number of domain embeddings; and
processing the domain embeddings using an output block of the annotation neural network to generate the plurality of mask-annotation pairs.

3. The method of claim 2, wherein the representation of the biological sequence comprises a respective initial per-position embedding of each position in the biological sequence that identifies a biological unit occupying the position in the biological sequence; and
wherein processing the representation of the biological sequence using an encoder block of the annotation neural network to generate a sequence of per-position embeddings that includes a respective per-position embedding for each position in the biological sequence comprises:
processing the initial per-position embeddings of the positions in the biological sequence using self-attention operations update the initial per-position embeddings of the positions in the biological sequence.

4. The method of claim 2, wherein processing the sequence of per-position embeddings for the biological sequence using the decoder block of the annotation neural network to generate the predefined number of domain embeddings comprises:
initializing the predefined number of domain embeddings; and
updating the domain embeddings by performing operations comprising:
self-attention operations over the domain embeddings to update the domain embeddings; and
cross-attention operations over the per-position embeddings for the biological sequence to update the domain embeddings.

5. The method of claim 2, wherein processing the domain embeddings using the output block of the annotation neural network comprises, for each domain embedding:
processing the domain embedding to generate a score distribution over a set of classes, wherein the set of classes comprises: (i) a plurality of domain classes, and (ii) a null class;
selecting a class from the set of classes based on the score distribution over the set of classes; and
determining whether to generate a mask-annotation pair from the domain embedding based on the selected class from the set of classes.

6. The method of claim 5, wherein selecting a class from the set of classes based on the score distribution over the set of classes comprises:
selecting a class having a highest score under the score distribution over the set of classes.

7. The method of claim 5, wherein for each domain embedding, determining whether to generate a mask-annotation pair from the domain embedding based on the selected class from the set of classes comprises:
determining that a mask-annotation pair should be generated from the domain embedding only if the selected class for the domain embedding is a domain class rather than the null class.

8. The method of claim 5, wherein for one or more domain embeddings:
selecting a class from the set of classes based on the score distribution over the set of classes comprises:
selecting a domain class for the domain embedding; and
processing the domain embedding using the output block of the annotation neural network further comprises:
processing the domain embedding and the sequence of per-position embeddings for the biological sequence to generate a score distribution over the biological sequence; and
generating a mask that identifies a domain of the biological sequence based on the score distribution over the biological sequence; and
outputting a mask-annotation pair that comprises: (i) the mask that identifies the domain, and (ii) an annotation that specifies the domain class.

9. The method of claim 8, wherein for one or more of the domain embeddings, processing the domain embedding and the sequence of per-position embeddings for the biological sequence to generate the score distribution over the biological sequence comprises, for each position in the biological sequence:
determining a score for the position in the score distribution based on a measure of similarity between: (i) the domain embedding, and (ii) the per-position embedding for the position.

10. The method of claim 9, wherein for each position in the biological sequence, determining the score for the position in the score distribution based on the measure of similarity between: (i) the domain embedding, and (ii) the per-position embedding for the position comprises:
computing an inner product between the domain embedding and the per-position embedding for the position.

11. The method of claim 2, wherein processing the domain embeddings using the output block of the annotation neural network comprises, for each domain embedding:
processing the domain embedding to generate a sequence of text; and
determining whether to generate a mask-annotation pair from the domain embedding based on the sequence of text.

12. The method of claim 11, wherein determining whether to generate the mask-annotation pair from the domain embedding based on the sequence of text comprises:
determining that a mask-annotation pair should be generated from the domain embedding only if the sequence of text indicates that the domain embedding corresponds to a domain in the biological sequence.

13. The method of claim 11, wherein for one or more domain embeddings, processing the domain embedding using the output block of the annotation neural network further comprises:
processing the domain embedding and the sequence of per-position embeddings for the biological sequence to generate a score distribution over the biological sequence; and
generating a mask that identifies a domain of the biological sequence based on the score distribution over the biological sequence; and
outputting a mask-annotation pair that comprises: (i) the mask that identifies the domain, and (ii) an annotation that specifies the sequence of text generated from the domain embedding.

14. The method of claim 1, wherein the plurality of mask-annotation pairs comprises one or more mask-annotation pairs that each include a respective mask that identifies a discontinuous domain that comprises multiple non-overlapping subsequences of the biological sequence.

15. The method of claim 1, wherein the plurality of mask-annotation pairs comprises multiple mask-annotation pairs that each include different masks but that share a same annotation.

16. The method of claim 1, wherein the biological sequence has a length of at least 16.

17. The method of claim 1, wherein the biological sequence comprises an amino acid sequence of a protein molecule.

18. The method of claim 1, wherein the biological sequence comprises a nucleic acid sequence of a deoxyribonucleic acid (DNA) molecule.

19. The method of claim 1, wherein the biological sequence comprises a nucleic acid sequence of a ribonucleic acid (RNA) molecule.

20. The method of claim 1, further comprising determining a function of the biological sequence based on the plurality of mask-annotation pairs.

21. The method of claim 1, wherein outputting the plurality of mask-annotation pairs comprises: storing the plurality of mask-annotation pairs in a memory; or transmitting the plurality of mask-annotation pairs over a data communications network; or providing a representation of the plurality of mask-annotation pairs on a display of a user device.

22. The method of claim 1, wherein the annotation neural network has been trained on a set of training examples by a machine learning training technique;
wherein the set of training examples each comprise: (i) a training biological sequence, and (ii) a plurality of target mask-annotation pairs for the for the training biological sequence; and
wherein training the annotation neural network on the set of training examples comprises, for each training example, training the annotation neural network to reduce a discrepancy between: (i) a plurality of predicted mask-annotation pairs generated by processing the training biological sequence using the annotation neural network, and (ii) the plurality of target mask-annotation pairs specified by the training example.

23. A system comprising:
one or more computers; and
one or more storage devices communicatively coupled to the one or more computers, wherein the one or more storage devices store instructions that, when executed by the one or more computers, cause the one or more computers to perform operations comprising:
obtaining data identifying a biological sequence;
processing a network input that comprises a representation of the biological sequence using an annotation neural network to generate a network output that defines a plurality of mask-annotation pairs, wherein each mask-annotation pair comprises:
(i) a mask that identifies a domain of the biological sequence; and
(ii) an annotation that characterizes the domain of the biological sequence; and
outputting the plurality of mask-annotation pairs.

24. One or more non-transitory computer storage media storing instructions that when executed by one or more computers cause the one or more computers to perform operations comprising:
obtaining data identifying a biological sequence;
processing a network input that comprises a representation of the biological sequence using an annotation neural network to generate a network output that defines a plurality of mask-annotation pairs, wherein each mask-annotation pair comprises:
(i) a mask that identifies a domain of the biological sequence; and
(ii) an annotation that characterizes the domain of the biological sequence; and
outputting the plurality of mask-annotation pairs.
